(19) United States
(12) Patent Application Publication
(10) Pub. No.: US 2012/0245556 A1
(43) Pub. Date: Sep. 27, 2012

(54) System, Method and Computer Program Product For Adjustment of Insulin Delivery in Diabetes Using Nominal Open-Loop Profiles

(75) Inventors: Boris P. Kovatchev, Charlottesville, VA (US); Giuseppe DeNicolao, Milano (IT); Lalo Magni, Vellezzo Bellini (IT); Chiara Dalla Man, Venezia (IT); Claudio Cobelli, Padova (IT)

(73) Assignee: UNIVERSITY OF VIRGINIA PATENT FOUNDATION, Charlottesville, VA (US)

(21) Appl. No.: 13/393,647

(22) PCT Filed: Aug. 31, 2010
(86) PCT No.: PCT/US10/47386

§ 371 Date: Jun. 15, 2012

Related U.S. Application Data
(60) Provisional application No. 61/238,807, filed on Sep. 1, 2009.

(51) Int. Cl. A61M 5/168

(52) U.S. Cl. 604/504; 604/500; 604/66

(57) ABSTRACT

A method, system and computer program product for correcting a nominal treatment strategy of a subject with diabetes. The method, system and computer program product may be configured for providing input whereby the input may include: open-loop therapy settings for the subject, data about glycemic state of the subject; and (optionally) data about meals and/or exercise of the subject. The method, system and computer program product may be configured for providing output, whereby the out-put may include an adjustment (correction) to the open-loop therapy settings for the subject for insulin delivery to the subject.

### RELATED APPLICATIONS

The present application claims priority from U.S. Provisional Application Ser. No. 61/238,807, filed Sep. 1, 2009, entitled “System, Method and Computer Program for Adjustment of Insulin Delivery in Diabetes Using Nominal Open-Loop Profiles,” the disclosure of which is hereby incorporated by reference herein in its entirety.

### BACKGROUND OF THE INVENTION

Importance of Glycemic Control in Diabetes: In health, blood glucose (BG) is tightly controlled by a hormonal network that includes the gut, liver, pancreas and brain, ensuring stable fasting BG levels (˜80-100 mg/dl) and transient postprandial glucose fluctuations. Diabetes is a combination of disorders characterized by absent or impaired insulin action, resulting in hyperglycemia. Intensive insulin and oral medication treatment to maintain nearly normal levels of glycemia markedly reduces chronic complications in both Type 1 (T1DM, See The Diabetes Control and Complications Trial Research Group. The effect of intensive treatment of diabetes on the development and progression of long-term complications of insulin-dependent diabetes mellitus. N Engl J Med 329: 978-986, 1993, of which is hereby incorporated by reference herein in its entirety) and Type 2 diabetes (T2DM, See UK Prospective Diabetes Study Group (UKPDS). Intensive blood-glucose control with sulphonylureas or insulin compared with conventional treatment and risk of complications in patients with type 2 diabetes. Lancet 352: 837-853, 1998, of which is hereby incorporated by reference herein in its entirety), but may cause a risk of potentially life-threatening severe hypoglycemia (SH). This SH results from imperfect insulin replacement, which may reduce warning symptoms and hormonal defenses, See Gold A E, Deary I J, Frier B M. Recurrent severe hypoglycaemia and cognitive function in type I diabetes. Diabet Med 10:503-508, 1993, of which is hereby incorporated by reference herein in its entirety. Consequently hypoglycemia has been identified as the primary barrier to optimal diabetes management. Iatrogenic hypoglycemia as a cause of hypoglycemia-associated autonomic failure in IDDM: A vicious cycle. Diabetes 41:255-260, 1992, of which is hereby incorporated by reference herein in its entirety].

### SUMMARY OF THE INVENTION

### Introduction

Control Strategies: Glucose control has been studied for now more than three decades and widely different solutions have been proposed, though it is only very recently that technology and algorithm have come together to enable glucose control elsewhere than in the intensive care unit (ICU) of a hospital.

Self Monitoring of Blood Glucose (SMBG)-Based Diabetes Management: The current management of diabetes typically uses Self Monitoring of Blood Glucose SMBG) to adjust the dosing of insulin delivered via injections or insulin pump.

Glucose is measured at infrequent (<5/day) and irregular time during the day and insulin is injected subcutaneously according to both these measures and the estimated amount of carbohydrates ingested. Depending on the treatment strategy the insulin is either injected continuously (basal rate) or discretely (boluses) via a pump, or only discretely, via injections containing both fast acting and long acting insulin. In both cases relation between the amount of insulin injected and the measured plasma glucose is determined by the care practitioner and the patient based on past experience and initial rule of thumbs (1800-rule and 450-rule). Insulin boluses are traditionally calculated in two phases: first, the amount of insulin is computed that is needed by a person to compensate for the carbohydrate content of an incoming meal. This is done by estimating the amount of carbohydrates to be ingested and multiplying by each person's insulin/carbohydrate ratio (CR). Second, the distance between actual blood glucose (BG) concentration and individual target level is calculated and the amount of insulin to reach the target is computed. This is done by multiplying the (BG—target) difference by individual insulin correction factor. Therefore a good assessment of each person's carbohydrate ratio (CR) and correction factor is critical for the optimal control of diabetes.

The Subcutaneous-Subcutaneous (SC-SC) Route: Since the advent of new technologies in glucose sensing and insulin infusion it is now possible to observe and act upon the glucose/insulin levels using real-time measurements: the sampling frequency of most meters being smaller or equal to 5 minutes. Therefore, increasing scientific and industrial effort is focused on the development of regulation systems (e.g. artificial pancreas) to control insulin delivery in people with diabetes. While these new technologies do open the way to both open and closed loop control of plasma glucose, they also suffer from drawbacks, such as, but not limited thereto:

The continuous sensors currently available experience delays estimated between 10 and 20 minutes.

The continuous sensors' accuracy is still lower than for example finger stick measurement (SMBG) and therefore none of the currently available sensors have been approved for ‘replacement’ by the Food & Drugs Administration (FDA), therefore precluding their use as such in clinical decision.

Subcutaneous (SC) injection of insulin imposes an additional actuation delay, the exogenous insulin being first transported from the injection site to the central vascular system and only then following the pathway of exogenous IV injected insulin.

Most recent control efforts have been focusing on the Subcutaneous-Subcutaneous (SC-SC) route as it is the most likely to be easily mass marketed and it relies on readily available technologies

An aspect of an embodiment of the present invention method, system and computer program product provides various approaches of operation of the Method for Adjustment of Insulin Delivery (AID). For example, some non-limiting examples may be as follows:

The AID receives blood glucose (BG) and insulin infusion data in real time from a continuous glucose monitor (CGM) and insulin pump (CSII), respectively;

The AID assesses continuously the risk for incipient hyperglycemic or hypoglycemic deviations from the pre-defined range and adjusts automatically, or suggests to the patient adjustments of, insulin delivery rate as appropriate; and

The AID permits and accounts for external insulin manipulation, e.g. basal rate, boluses, or insulin pump shutoff initiated by the patient.

An aspect of an embodiment of the present invention provides a method for correcting a nominal treatment strategy of a subject with diabetes. The method may comprise: providing input, whereby the input may include: open-loop therapy settings for the subject; data about glycemic state of the subject; and (optionally) data about meals and/or exercise of the subject. The method may comprise providing output, whereby the output may include an adjustment (correction) to the open-loop therapy settings for the subject for insulin delivery to the subject.

An aspect of an embodiment of the present invention provides a system for correcting a nominal treatment strategy of a subject with diabetes. The system may comprise: an open loop therapy module, wherein the open loop therapy module provides open-loop therapy settings for the subject; a glucose monitor, wherein the glucose monitor provides data about the glycemic state of the subject; an insulin pump, wherein the insulin pump provides data about the glycemic state of the subject; optionally, a meal and/or exercise module, wherein the optional meal/exercise module provides data about meals and/or exercise of the subject; and an adjustment of insulin delivery (AID) module, wherein the AID module provides an adjustment to the open-loop therapy settings for the subject for insulin delivery to the subject.

An aspect of an embodiment of the present invention provides a computer program product comprising a computer useable medium having a computer program logic for enabling at least one processor in a computer system for correcting a nominal treatment strategy of a subject with diabetes. The computer program logic may be comprised of or configured for: providing input, whereby the input may include: open-loop therapy settings for the subject; data about glycemic state of the subject; and (optionally) data about meals and/or exercise of the subject. The logic may be comprised of or configured for: providing output, whereby the output may include an adjustment (correction) to the open-loop therapy settings for the subject for insulin delivery to the subject.

These and other objects, along with advantages and features of various aspects of embodiments of the invention disclosed herein, will be made more apparent from the description, drawings and claims that follow.

### BRIEF DESCRIPTION OF THE DRAWINGS

The foregoing and other objects, features and advantages of the present invention, as well as the invention itself, will be more fully understood from the following description of preferred embodiments, when read together with the accompanying drawings

The accompanying drawings, which are incorporated into and form a part of the instant specification, illustrate several aspects and embodiments of the present invention and, together with the description herein, serve to explain the principles of the invention. The drawings are provided only for the purpose of illustrating select embodiments of the invention and are not to be construed as limiting the invention.

FIG. 1 schematically illustrates the adjustment function associated with the AID system and method of an aspect of and embodiment of the present invention.

FIG. 2 is a schematic block diagram for a system or related method of an aspect of an embodiment of the present invention in whole or in part.

FIG. 3 graphically presents the design (or exercise protocol) of clinical trials of an embodiment of the present invention.

FIG. 4 graphically presents the results from the first of these clinical trials.

FIG. 5 graphically presents summary data results from these studies of FIGS. 3 and 4:

### DETAILED DESCRIPTION OF THE INVENTION

An exemplary concept of an aspect of an embodiment of the present invention AID method (and related system and computer program product) is the notion of nominal open-loop profile. The nominal open-loop profile is a treatment strategy determined for each person in advance from patient records or observation, which is believed to be routine or typical for this individual. As described below, the AID method (and related system and computer program product) acts by introducing corrections (or adjustments) to the nominal treatment strategy. This is a fundamental difference from typical closed-loop control algorithm where a target to be followed is considered and the open-loop nominal profile knowledge is lost.

Specifically, the nominal open-loop profile is determined from the outcomes of open-loop treatment strategy as follows:

uₒ(k)=fₒ(Mₒ(k),Eₒ(k))

yₒ(k)=gₒ(Mₒ(k),Eₒ(k))

where k indicates the current time instant, uₒ(k) is the nominal insulin delivery, yₒ(k) is the nominal subcutaneous glucose concentration, Mₒ(k) is a vector with past and future (if available) meals considered in the computation of the nominal open-loop profile and Eₒ(k) is a vector with past and future (if available) physical exercise values or other possible disturbance information considered in the computation of the nominal open-loop profile. The functions fₒ and gₒ are computational rules that may either rely on a mathematical model of glucose metabolism or reflect medical expertise possibly accounting also for historical records. In both cases, uₒ(k) is the insulin delivery that best accommodates available meal, exercise, and disturbance information, based either on mathematical models or medical expertise. On the other hand, yₒ(k) is the subcutaneous concentration that is expected under this open-loop control, evaluated on the basis of either mathematical models or medical expertise. In the following, y(k) denotes the actual CGM measurement.

The AID method (and related system and computer program product) is based on, but not limited thereto, the nominal open-loop profile and has a principal goal to maintain the open-loop behaviour in conditions close to nominal. It adjusts the open-loop strategy only when the observed patient's behavior differs from the nominal one, for instance due to changes in the patient parameters or external disturbances different from the nominal ones. The AID method relies on an observation horizon Nₒ, which should be long enough to assess and predict the extent of possible departures of patient's behavior from the nominal one. With this understanding, the AID control law is given by

u(k)=uₒ(k)+uₐ (k)

uₐ(k)=fₐ(M(k),Mₒ(k),E(k),Eₒ(k),Y(k),Yₒ(k),U(k), Uₒ(k))

where u(k) is the actual delivered insulin, uₐ(k) is the insulin adjustment term, M(k) is a vector with past and future (if available) meals and E(k) is a vector with past and future (if available) physical exercise values or other possible disturbance information and

Y(k)=[y(k) y(k−1) . . . y(k−Nₒ)]

Yₒ(k)=[yₒ(k) yₒ(k−1) . . . yₒ(k−Nₒ)]

U(k)=[u(k−1) u(k−2) . . . u(k−Nₒ)]

Uₒ(k)=[uₒ(k−1) uₒ(k−2) . . . uₒ(k−Nₒ)]

The adjustment function fₐ is the core of AID: it evaluates nominal insulin and CGM profiles in order to compute the insulin adjustment term. The principal characteristic of the AID control law can then be formulated as:

f(M(k),Mₒ(k),E(k),Eₒ(k),Y(k),Yₒ(k),U(k),Uₒ(k))=0 if Y(k)=Yₒ(k) and M(k)=Mₒ(k), E(k)=Eₒ(k) U(k)=Uₒ(k)

In other words, AID adjusts the nominal open-loop insulin delivery, only if the observed patient's behavior or blood glucose fluctuations differ from nominal. The adjustment function fₐ is designed following a control-to-range objective: it has to keep and possibly bring back the actual CGM y(k) within a range of the nominal CGM yₒ(k). This objective can be achieved by means of largely different algorithms including, but not limited to, regulators inspired to Model Predictive Control principles.

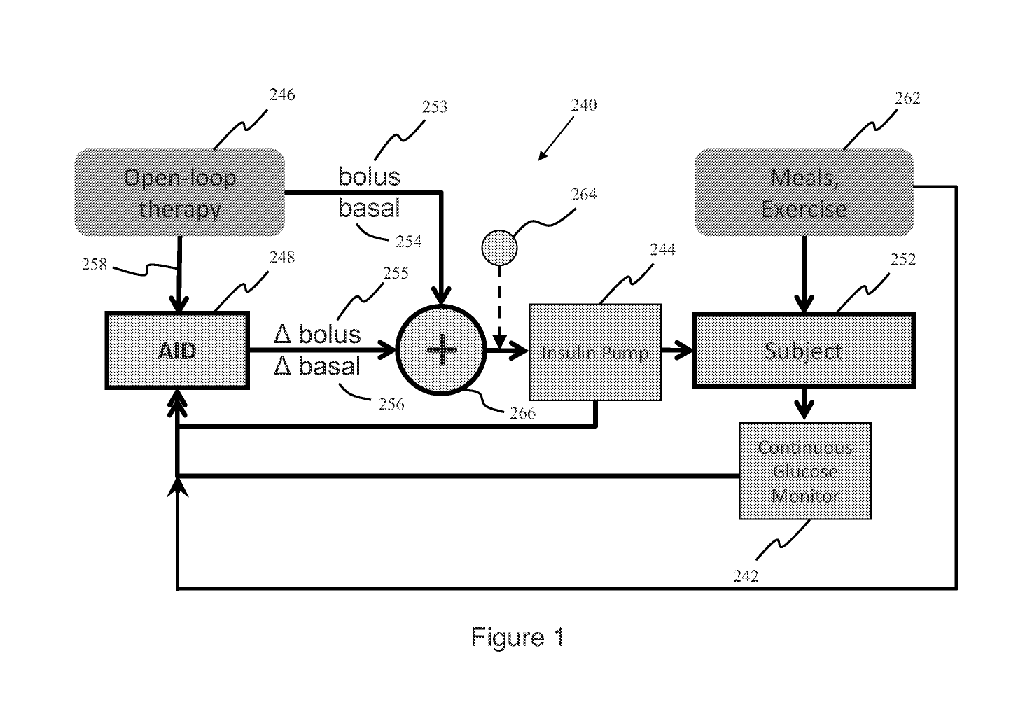

This adjustment function of an embodiment of the system 240 is presented in FIG. 1: The insulin bolus information and/or insulin basal rate information (e.g., pre-set basal rate and predetermined insulin bolus amounts) from any open-loop therapy module 246 is sent 258 to the AID module 248. The AID module 248 computes appropriate corrections (adjustments) to this information using input from a continuous glucose monitor 242, insulin pump 244, and meal/exercise module 262. These corrections (adjustments) 255, 256 are then added by the summation module 266 to the original basal rate 253 /boluses 254 (e.g., pre-set basal rate and predetermined insulin bolus amounts) and the resulting amount of insulin is delivered to the insulin pump 244 intended for the subject 252. It may be noted that this amount could be positive if more insulin is needed, or negative, if the originally prescribed insulin were too much. In this embodiment, the AID module 248 is an open loop-informed linear model-predictive controller, whereby real-time optimization is not needed; only one parameter, q, requires individual tuning based on carbohydrate ratio (CR) and basal insulin; and the sample frequency is about every 15 minutes. It should be appreciated that the sample frequency may be approximately every 5 to 20 minutes. It should be appreciated that the sample frequency may be less frequent or more frequent as desired or required.

It should be appreciated that the time-interval for obtaining the samples may be: about four times per hour; about once per hour; more than once per hour; or less than once per hour.

Moreover, the output from the summation module 266 that is intended to be delivered to the insulin pump 244 that in turn is intended for the subject 252 may be optionally subjected to a safety supervision system 264. The related embodiment and approach pertaining to the safety supervision system 264 is disclosed by the Applicant in PCT International Patent Application Serial No. PCT/US2010/025405, filed Feb. 25, 2010, entitled “Method, System and Computer Program Product for CGM-Based Prevention of Hypoglycemia via Hypoglycemia Risk Assessment and Smooth Reduction Insulin Delivery,” of which is hereby incorporated by reference herein in its entirety. It should be appreciated that the referenced embodiment and approach of the safety supervision system may be implemented with the present disclosure/embodiments.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It should be appreciated that the subject may be any applicable human patient, for example.

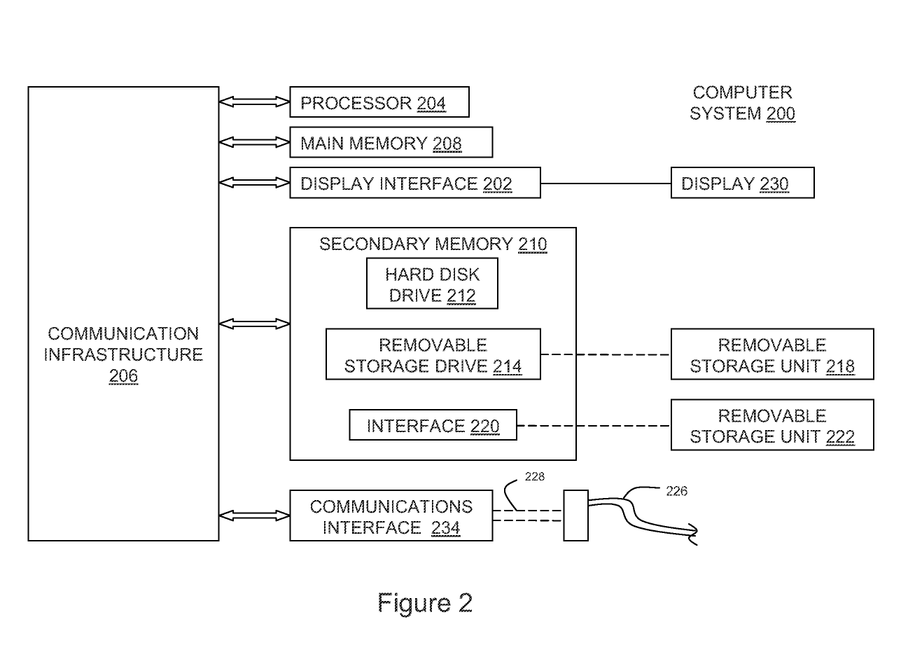

The modules and components of FIGS. 1-2 may be transmitted to the appropriate or desired computer networks in various locations and sites (local and/or remote) as desired or required.

The modules and components of FIGS. 1-2 may be transmitted to the appropriate or desired computer networks in various locations and sites (local and/or remote) via the desired or required communication links.

It should be appreciated that any of the components/modules discussed in FIGS. 1-2 may be integrally contained within one or more housings or separated and/or duplicated in different housings. Similarly, any of the components and modules discussed in FIGS. 1-2 may be duplicated more than once. Moreover, various components and modules may be adapted to replace another component or module to perform the intended function.

It should also be appreciated that any of the components/modules present in FIGS. 11-13 may be in direct or indirect communication with any of the other components/modules.

It should be appreciated that the modules and components as depicted in FIGS. 1-2 may be implemented with any location, person, staff, physician, caregiver, system, device or equipment at any healthcare provider, hospital, clinic, university, vehicle, trailer, or home, as well as any other location, premises, or organization as desired or required.

Turning to FIG. 2, FIG. 2 is a functional block diagram for a computer system 200 for implementation of an exemplary embodiment or portion of an embodiment of present invention. For example, a method or system of an embodiment of the present invention may be implemented using hardware, software or a combination thereof and may be implemented in one or more computer systems or other processing systems, such as personal digit assistants (PDAs) equipped with adequate memory and processing capabilities. In an example embodiment, the invention was implemented in software running on a general purpose computer 200 as illustrated in FIG. 2. The computer system 200 may includes one or more processors, such as processor 204. The Processor 204 is connected to a communication infrastructure 206 (e.g., a communications bus, cross-over bar, or network). The computer system 200 may include a display interface 202 that forwards graphics, text, and/or other data from the communication infrastructure 206 (or from a frame buffer not shown) for display on the display unit 230. Display unit 230 may be digital and/or analog.

The computer system 200 may also include a main memory 208, preferably random access memory (RAM), and may also include a secondary memory 210. The secondary memory 210 may include, for example, a hard disk drive 212 and/or a removable storage drive 214, representing a floppy disk drive, a magnetic tape drive, an optical disk drive, a flash memory, etc. The removable storage drive 214 reads from and/or writes to a removable storage unit 218 in a well known manner. Removable storage unit 218, represents a floppy disk, magnetic tape, optical disk, etc. which is read by and written to by removable storage drive 214. As will be appreciated, the removable storage unit 218 includes a computer usable storage medium having stored therein computer software and/or data.

In alternative embodiments, secondary memory 210 may include other means for allowing computer programs or other instructions to be loaded into computer system 200. Such means may include, for example, a removable storage unit 222 and an interface 220. Examples of such removable storage units/interfaces include a program cartridge and cartridge interface (such as that found in video game devices), a removable memory chip (such as a ROM, PROM, EPROM or EEPROM) and associated socket, and other removable storage units 222 and interfaces 220 which allow software and data to be transferred from the removable storage unit 222 to computer system 200.

The computer system 200 may also include a communications interface 224. Communications interface 224 allows software and data to be transferred between computer system 200 and external devices. Examples of communications interface 224 may include a modem, a network interface (such as an Ethernet card), a communications port (e.g., serial or parallel, etc.), a PCMCIA slot and card, a modem, etc. Software and data transferred via communications interface 224 are in the form of signals 228 which may be electronic, electromagnetic, optical or other signals capable of being received by communications interface 224. Signals 228 are provided to communications interface 224 via a communications path (i.e., channel) 226.

Channel 226 (or any other communication means or channel disclosed herein) carries signals 228 and may be implemented using wire or cable, fiber optics, blue tooth, a phone line, a cellular phone link, an RF link, an infrared link, wireless link or connection and other communications channels.

In this document, the terms “computer program medium” and “computer usable medium” are used to generally refer to media or medium such as various software, firmware, disks, drives, removable storage drive 214, a hard disk installed in hard disk drive 212, and signals 228. These computer program products (“computer program medium” and “computer usable medium”) are means for providing software to computer system 200. The computer program product may comprise a computer useable medium having computer program logic thereon. The invention includes such computer program products. The “computer program product” and “computer useable medium” may be any computer readable medium having computer logic thereon.

Computer programs (also called computer control logic or computer program logic) are may be stored in main memory 208 and/or secondary memory 210. Computer programs may also be received via communications interface 224. Such computer programs, when executed, enable computer system 200 to perform the features of the present invention as discussed herein. In particular, the computer programs, when executed, enable processor 204 to perform the functions of the present invention. Accordingly, such computer programs represent controllers of computer system 200.

In an embodiment where the invention is implemented using software, the software may be stored in a computer program product and loaded into computer system 200 using removable storage drive 214, hard drive 212 or communications interface 224. The control logic (software or computer program logic), when executed by the processor 204, causes the processor 204 to perform the functions of the invention as described herein.

In another embodiment, the invention is implemented primarily in hardware using, for example, hardware components such as application specific integrated circuits (ASICs). Implementation of the hardware state machine to perform the functions described herein will be apparent to persons skilled in the relevant art(s).

In yet another embodiment, the invention is implemented using a combination of both hardware and software.

In an example software embodiment of the invention, the methods described above may be implemented in SPSS control language or C++ programming language, but could be implemented in other various programs, computer simulation and computer-aided design, computer simulation environment, MATLAB, or any other software platform or program, windows interface or operating system (or other operating system) or other programs known or available to those skilled in the art.

An aspect of various embodiments of the present invention may provide a number of advantages. For example, but not limited thereto, the alternative artificial pancreas device (and related method) provides the ability to exploit the nominal open-loop profile of the specific patient so as to adjust insulin delivery in an optimized and personalized way. Other closed-loop devices do not fully exploit the knowledge of the individual open-loop therapy.

Further, the AID method (and related system and computer program product) can be the basis for the design of artificial pancreas devices that exploit both the medical knowledge intrinsic in a well calibrated open-loop nominal therapy and the robustness properties coming from intelligent and timely use of continuous glucose monitoring.

### EXAMPLES

### Experimental Results Set No. 1

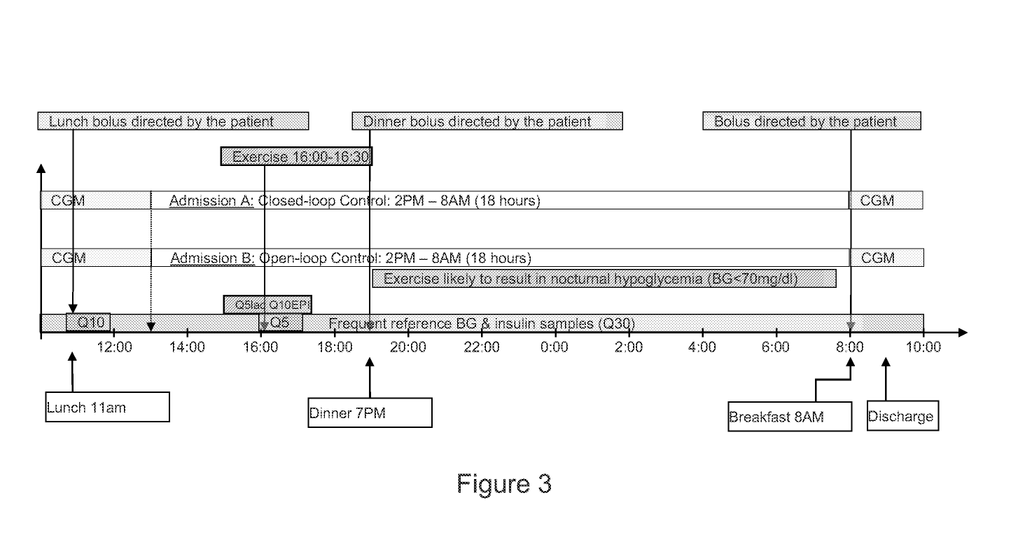

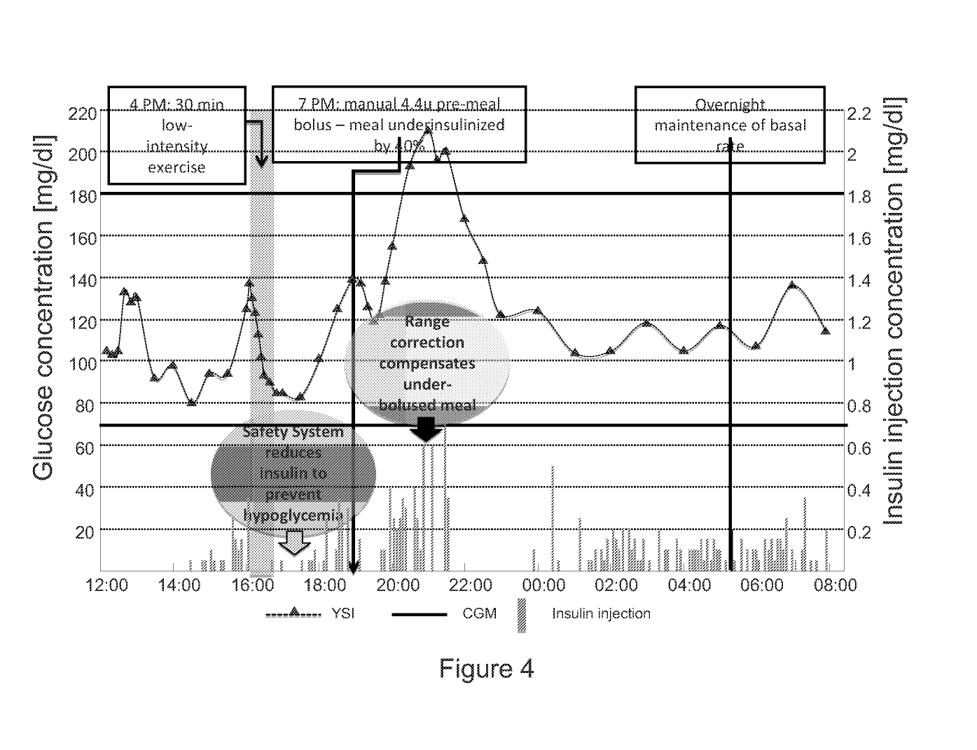

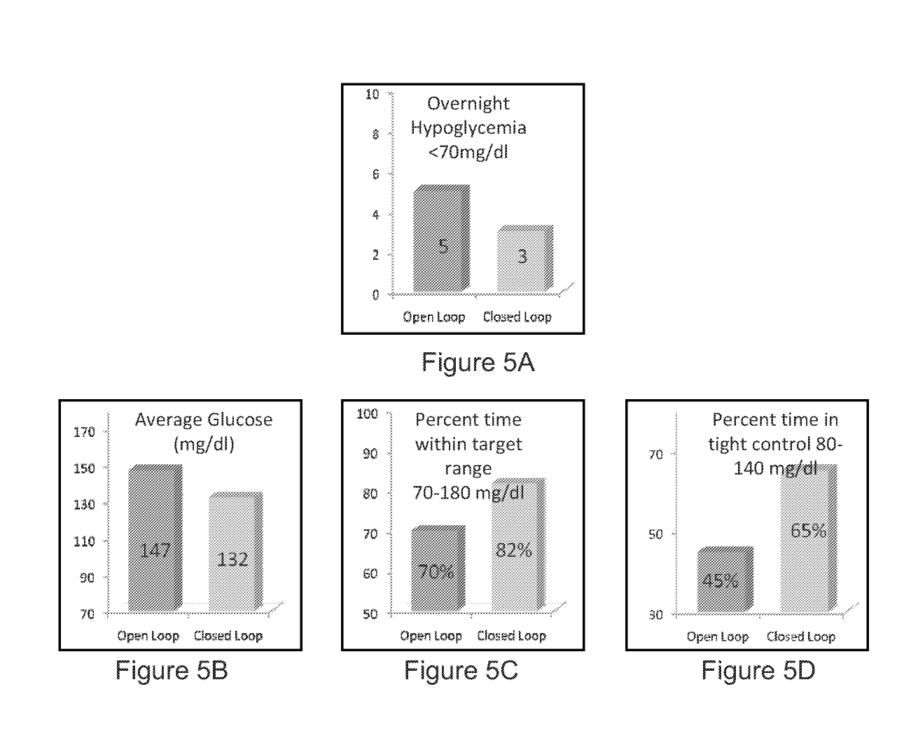

Practice of an aspect of an embodiment (or embodiments) of the invention will be still more fully understood from the following experimental results, which are presented herein for illustration only and should not be construed as limiting the invention in any way. An implementation of an aspect of an embodiment of the present invention was tested in clinical trials that enrolled patients with type 1 diabetes (T1DM). FIG. 3 graphically presents the design (e.g., exercise protocol) of these clinical trials: each patient (subject) was tested on two different days: standard-treatment open-loop control and closed-loop control using the AID method and optionally a safety supervision system, which is the subject of a separate invention, embodiment and approach (See PCT International Patent Application Serial No. PCT/US2010/025405, filed Feb. 25, 2010, entitled “Method, System and Computer Program Product for CGM-Based Prevention of Hypoglycemia via Hypoglycemia Risk Assessment and Smooth Reduction Insulin Delivery,” of which is hereby incorporated by reference herein in its entirety. The study included physical activity challenges, which are typically a major cause for hypoglycemia in diabetes. FIG. 4 graphically presents the results from the first of these clinical trials : it is evident that the AID method achieved excellent glucose control, keeping the subject within target range (70-180 mg/dl) for over 90% of the time. FIG. 4 also illustrates the concept of integrating the AID method as part of a closed-loop control system: combined with the safety supervision, all potential hypoglycemic episodes were prevented. Finally, FIG. 5 graphically presents summary data from these studies, that included 6 adults with Type 1 diabetes (T1DM) and it covered the full Control-to-Range (CTR), Range Control Module and Safety Supervisor System. It is evident that, compared to optimal open-loop control done under physician's supervision, the AID method resulted in less hypoglycemia overnight (FIG. 5(A)), better average glucose (FIG. 5(B)), and higher percentage of time spent within the desired target ranges, for example 70-180 mg/dl and 80-140 mg/dl (FIGS. 5(C) and 5(D)), respectively).

The devices, systems, compositions, computer program products, and methods of various embodiments of the invention disclosed herein may utilize aspects disclosed in the following references, applications, publications and patents and which are hereby incorporated by reference herein in their entirety:

The devices, systems, and computer program products, and methods of various embodiments of the invention disclosed herein may utilize aspects disclosed in the following references, applications, publications and patents and which are hereby incorporated by reference herein in their entirety:

A. International Patent Application Serial No. PCT/US2010/025405, entitled “Method, System and Computer Program Product for CGM-Based Prevention of Hypoglycemia via Hypoglycemia Risk Assessment and Smooth Reduction Insulin Delivery,” filed Feb. 25, 2010.

B. International Patent Application Serial No. PCT/US2009/065725, filed Nov. 24, 2009,entitled “Method, System, and Computer Program Product for Tracking of Blood Glucose Variability in Diabetes from Data,”

C. PCT/US2008/082063, entitled “Model Predictive Control Based Method for Closed-Loop Control of Insulin Delivery in Diabetes Using Continuous Glucose Sensing”, filed Oct. 31, 2008.

D. PCT/US2008/069416, entitled “Method, System and Computer Program Product for Evaluation of Insulin Sensitivity, Insulin/Carbohydrate Ratio, and Insulin Correction Factors in Diabetes from Self-Monitoring Data”, filed Jul. 8, 2008.

E. PCT/US2008/067725, entitled “Method, System and Computer Simulation Environment for Testing of Monitoring and Control Strategies in Diabetes,” filed Jun. 20, 2008.

F. PCT/US2008/067723, entitled “LQG Artificial Pancreas Control System and Related Method”, filed on Jun. 20, 2008.

G. U.S. Ser. No. 12/516,044, filed May 22, 2009, entitled “Method, System, and Computer Program Product for the Detection of Physical Activity by Changes in Heart Rate, Assessment of Fast Changing Metabolic States, and Applications of Closed and Open Control Loop in Diabetes;”

H. PCT/US2007/085588 not yet published filed Nov. 27, 2007, entitled “Method, System, and Computer Program Product for the Detection of Physical Activity by Changes in Heart Rate, Assessment of Fast Changing Metabolic States, and Applications of Closed and Open Control Loop in Diabetes;”

I. U.S. Ser. No. 11/943,226, filed Nov. 20, 2007, entitled “Systems, Methods and Computer Program Codes for Recognition of Patterns of Hyperglycemia and Hypoglycemia, Increased Glucose Variability, and Ineffective Self-Monitoring in Diabetes;”

J. U.S. patent application Ser. No. 11/578,831, filed Oct. 18, 2006 entitled “Method, System and Computer Program Product for Evaluating the Accuracy of Blood Glucose Monitoring Sensors/Devices”.

K. PCT International Application Serial No. PCT/US2005/013792, filed Apr. 21, 2005, entitled “Method, System, and Computer Program Product for Evaluation of the Accuracy of Blood Glucose Monitoring Sensors/Devices;”

L. PCT International Application Serial No. PCT/US01/09884, filed Mar. 29 2001, entitled “Method, System, and Computer Program Product for Evaluation of Glycemic Control in Diabetes Self-Monitoring Data;”

M. U.S. Pat. No. 7,025,425 B2 issued Apr. 11, 2006, entitled “Method, System, and Computer Program Product for the Evaluation of Glycemic Control in Diabetes from Self-Monitoring Data;”

N. U.S. patent application Ser. No. 11/305,946 filed Dec. 19, 2005 entitled “Method, System, and Computer Program Product for the Evaluation of Glycemic Control in Diabetes from Self-Monitoring Data” (Publication No. 2006/0094947);

O. PCT International Application Serial No. PCT/US2003/025053, filed Aug. 8, 2003, entitled “Method, System, and Computer Program Product for the Processing of Self-Monitoring Blood Glucose (SMBG) Data to Enhance Diabetic Self-Management;”

P. U.S. patent application Ser. No. 10/524,094 filed Feb. 9, 2005 entitled “Managing and Processing Self-Monitoring Blood Glucose” (Publication No. 2005/214892);

Q. U.S. Ser. No. 12/065,257, filed Aug. 29, 2008, entitled “Accuracy of Continuous Glucose Sensors;”

R. PCT International Application Serial No PCT/US2006/033724, filed Aug. 29, 2006, entitled “Method for Improvising Accuracy of Continuous Glucose Sensors and a Continuous Glucose Sensor Using the Same;”

S. U.S. Ser. No. 12/159,891, filed Jul. 2, 2008, entitled “Method, System and Computer Program Product for Evaluation of Blood Glucose Variability in Diabetes from Self-Monitoring Data;”

T. PCT International Application No. PCT/US2007/000370, filed Jan. 5, 2007, entitled “Method, System and Computer Program Product for Evaluation of Blood Glucose Variability in Diabetes from Self-Monitoring Data;”

U. U.S. patent application Ser. No. 11/925,689 and PCT International Patent Application No. PCT/US2007/082744, both filed Oct. 26, 2007, entitled “For Method, System and Computer Program Product for Real-Time Detection of Sensitivity Decline in Analyte Sensors;”

V. U.S. Ser. No. 10/069,674, filed Feb. 22, 2002, entitled “Method and Apparatus for Predicting the Risk of Hypoglycemia;”

W. PCT International Application No. PCT/US00/22886, filed Aug. 21, 2000, entitled “Method and Apparatus for Predicting the Risk of Hypoglycemia;”

X. U.S. Pat. No. 6,923,763 B1, issued Aug. 2, 2005, entitled “Method and Apparatus for Predicting the Risk of Hypoglycemia;”

Y. U.S. Patent Application No. US 2004/0254434 A1, “Glucose Measuring Module and “Insulin Pump Combination”, Dec. 16, 2004.

Z. U.S. Patent Application Publication No. US 2009/00697456 A1, Estes, et al., “Operating an Infusion Pump System”, Mar. 12, 2009.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the following claims, including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

We claim:

1. A method for correcting a nominal treatment strategy of a subject with diabetes, said method comprising:
providing input, wherein:
said input comprises:
open-loop therapy settings for the subject, and
data about glycemic state of the subject; and
said input optionally comprises:
data about meals and/or exercise of the subject; and
providing output, wherein:
said output comprises:
an adjustment to said open-loop therapy settings for the subject for insulin delivery to the subject.

2. The method of claim 1, wherein said adjustment being provided by an adjustment of insulin delivery (AID) module.

3. The method of claim 2, further comprising:
adding said adjustment from said AID module to said open-loop therapy settings for the subject for said insulin delivery to the subject.

4. The method of claim 1, wherein said open-loop therapy settings comprises one of the following:
pre-set basal rate;
predetermined insulin bolus amounts; or
pre-set basal rate and predetermined insulin bolus amounts.

5. The method of claim 1, wherein said glycemic state data comprises insulin pump data and blood glucose monitor data.

6. The method of claim 1, wherein said glycemic data is obtained during certain timing, wherein said timing comprises at least one of the following: episodic data, time-interval data, or periodic data.

7. The method of claim 6, wherein said time-interval for said obtaining comprises: about four times per hour.

8. The method of claim 6, wherein said time-interval for said obtaining comprises: about once per hour.

9. The method of claim 6, wherein said time-interval for said obtaining comprises: more than once per hour.

10. The method of claim 6, wherein said time-interval for said obtaining comprises: less than once per hour.

11. The method of claim 6, wherein said episodic data comprise data obtained from Self Monitoring of Blood Glucose (SMBG) or other types of episodic data.

12. The method of claim 1, wherein said optional meal data comprises at least one of the following:
time of meals, carbohydrate amount, or composition of meals.

13. The method of claim 1, wherein if said optional input is pursued in claim 1, said optional exercise data comprises at least one of the following:
timing of exercise, duration of exercise, and intensity of exercise.

14. The method of claim 1, further comprising:
adding said adjustment to said open-loop therapy settings for the subject for said insulin delivery to the subject.

15. The method of claim 1, wherein said adjustment being provided in accordance with the following equations:
u(k)=uₒ(k)+uₐ(k)
uₐ(k)=fₐ(M(k),Mₒ(k), E(k), Eₒ(k),Y(k),Yₒ(k),U(k),Uₒ(k))
where u(k) is the actual delivered insulin;
uₐ(k) is the insulin adjustment term;
Y(k)=[y(k)y(k−1) . . . y(k−Nₒ)]
Yₒ(k)=[yₒ(k)yₒ(k−1) . . . yₒ(k−Nₒ)]
U(k)=[u(k−1) u(k−2) . . . u(k−Nₒ)]
Uₒ(k)=[uₒ(k−1) uₒ(k−2) . . . uₒ(k−Nₒ)]
fₐ is the adjustment function;
uₒ(k)=fₒ(Mₒ(k), Eₒ(k))
yₒ(k)=gₒ(Mₒ(k), Eₒ(k))
where k indicates the current time instant;
uₒ(k) is the nominal insulin delivery;
yₒ(k) is the nominal subcutaneous glucose concentration,;
if said optional input is pursued in claim 1, then Mₒ(k) is a vector with past and future meals considered in the computation of the nominal open-loop profile;
if said optional input is pursued in claim 1, then M(k) is a vector with past and future meals;
if said optional input is pursued in claim 1, then Eₒ(k) is a vector with past and future physical exercise values or other possible disturbance information considered in the computation of the nominal open-loop profile;
if said optional input is pursued in claim 1, then E(k) is a vector with past and future physical exercise values or other possible disturbance information
functions fₒ and gₒ are computational rules that may either rely on a mathematical model of glucose metabolism or reflect medical expertise possibly accounting also for historical records;
uₒ(k) is the insulin delivery that best accommodates available meal, exercise, and disturbance information, based either on mathematical models or medical expertise;
yₒ(k) is the subcutaneous concentration that is expected under this open-loop control, evaluated on the basis of either mathematical models or medical expertise; and
y(k) denotes the actual continuous glucose monitoring (CGM) measurement.

16. A system for correcting a nominal treatment strategy of a subject with diabetes, said system comprising:
an open loop therapy module, wherein said open loop therapy module provides open-loop therapy settings for the subject;
a glucose monitor, wherein said glucose monitor provides data about the glycemic state of the subject;
an insulin pump, wherein said insulin pump provides data about the glycemic state of the subject;
optionally, a meal and/or exercise module, wherein said optional meal/exercise module provides data about meals and/or exercise of the subject; and
an adjustment of insulin delivery (AID) module, wherein said AID module provides an adjustment to said open-loop therapy settings for the subject for insulin delivery to the subject.

17. The system of claim 16, wherein said open-loop therapy settings comprises one of the following:
pre-set basal rate;
predetermined insulin bolus amounts; or
pre-set basal rate and predetermined insulin bolus amounts.

18. The system of claim 16, wherein said glycemic state data comprises insulin pump data and blood glucose monitor data.

19. The system of claim 18, wherein said glycemic data is obtained during certain timing, wherein said timing comprises at least one of the following: episodic data, time-interval data, or periodic data.

20. The system of claim 19, wherein said time-interval for said obtaining comprises: about four times per hour.

21. The system of claim 19, wherein said time-interval for said obtaining comprises: about once per hour.

22. The system of claim 19, wherein said time-interval for said obtaining comprises: more than once per hour.

23. The system of claim 19, wherein said time-interval for said obtaining comprises: less than once per hour.

24. The system of claim 19, wherein said episodic data comprise data obtained from Self Monitoring of Blood Glucose SMBG) or other types of episodic data.

25. The system of claim 16, wherein if said optional meal and/or exercise module is present in claim 16, said meal module comprises at least one of the following:
time of meals, carbohydrate amount, or composition of meals.

26. The system of claim 16, wherein if said optional meal and/or exercise module is present in claim 16, said optional exercise data comprises at least one of the following:
timing of exercise, duration of exercise, and intensity of exercise.

27. The system of claim 16, further comprising:
a summation module, wherein said summation module for adding said adjustment from said AID module to said open-loop therapy settings for the subject for said insulin delivery to the subject.

28. The system of claim 16, wherein said adjustment being provided in accordance with the following equations:
u(k)=uₒ(k)+uₐ(k)
uₐ(k)=fₐ(M(k),Mₒ(k), E(k), Eₒ(k),Y(k),Yₒ(k),U(k),Uₒ(k))
where u(k) is the actual delivered insulin;
uₐ (k) is the insulin adjustment term;
Y(k)=[y(k)y(k−1) . . . y(k−Nₒ)]
Yₒ(k)=[yₒ(k)yₒ(k−1) . . . yₒ(k−Nₒ)]
U(k)=[u(k−1) u(k−2) . . . u(k−Nₒ)]
Uₒ(k)=[uₒ(k−1) uₒ(k−2) . . . uₒ(k−Nₒ)]
fₐ is the adjustment function;
uₒ(k)=fₒ(Mₒ(k), Eₒ(k))
yₒ(k)=gₒ(Mₒ(k), Eₒ(k))
where k indicates the current time instant;
uₐ(k) is the nominal insulin delivery;
yₒ(k) is the nominal subcutaneous glucose concentration,;
if said optional meal and/or exercise module is present in claim 16, then Mₒ(k) is a vector with past and future meals considered in the computation of the nominal open-loop profile;
if said optional meal and/or exercise module is present in claim 16, then M(k) is a vector with past and future meals;
if said optional meal and/or exercise module is present in claim 16, then Eₒ(k) is a vector with past and future physical exercise values or other possible disturbance information considered in the computation of the nominal open-loop profile;
if said optional meal and/or exercise module is present in claim 16, then E(k) is a vector with past and future physical exercise values or other possible disturbance information
functions fₒ and gₒ are computational rules that may either rely on a mathematical model of glucose metabolism or reflect medical expertise possibly accounting also for historical records;
uₒ(k) is the insulin delivery that best accommodates available meal, exercise, and disturbance information, based either on mathematical models or medical expertise;
yₒ(k) is the subcutaneous concentration that is expected under this open-loop control, evaluated on the basis of either mathematical models or medical expertise; and
y(k) denotes the actual continuous glucose monitoring (CGM) measurement.

29. A computer program product comprising a computer useable medium having a computer program logic for enabling at least one processor in a computer system for correcting a nominal treatment strategy of a subject with diabetes, said computer program logic comprising:
providing input, wherein:
said input comprises:
open-loop therapy settings for the subject, and
data about glycemic state of the subject; and
said input optionally comprises:
data about meals and/or exercise of the subject; and
providing output, wherein:
said output comprises:
an adjustment to said open-loop therapy settings for the subject for insulin delivery to the subject.

30. The computer program product of claim 29, wherein said processor is configured to be in communication with one or more of the following:
an open loop therapy module,
a glucose monitor,
an insulin pump,
optionally, a meal and/or exercise module, or
an adjustment of insulin delivery (AID) module.

31. The computer program product of claim 29, wherein said processor is configured to be in communication with one or more of the following:
a memory device;
a display interface;
a display interface;
a communications interface; or
a communications path.
